(19) United States
(12) Patent Application Publication
(10) Pub. No.: US 2019/0191988 A1
(43) Pub. Date: Jun. 27, 2019

(54) SCREENING METHOD FOR AUTOMATED DETECTION OF VISION-DEGENERATIVE DISEASES FROM COLOR FUNDUS IMAGES

(71) Applicant: Spect Inc., Palo Alto, CA (US)

(72) Inventor: Rishab GARGEYA, Saratoga, CA (US)

(21) Appl. No.: 16/288,308

(22) Filed: Feb. 28, 2019

Related U.S. Application Data
(63) Continuation of application No. PCT/US2017/049984, filed on Sep. 2, 2017.
(60) Provisional application No. 62/383,333, filed on Sep. 2, 2016.

(51) Int. Cl. A61B 3/00; A61B 3/12; A61B 3/14; A61B 5/00; G06T 7/00

(52) U.S. Cl. CPC A61B 3/0025; A61B 3/1241; A61B 3/14; A61B 5/7267; A61B 5/7275; G06T 2207/20081; A61B 5/725; A61B 5/6898; G06T 7/0014; G06T 2207/30041; G06T 2207/20084; A61B 5/7282

(57) ABSTRACT

Disclosed herein are methods and apparatuses for detecting vision-degenerative diseases. A first method comprises obtaining a dataset of fundus images, using a custom deep learning network to process the dataset of fundus images, and providing, as an output, a function for use in diagnosing a vision-degenerative disease. A computing device comprises memory storing a representation of the function produced by the first method, and one or more processors configured to use the function to assist in the diagnosis of the vision-degenerative disease. A second method is for determining a likelihood that a patient's eye has a vision-degenerative disease, the method comprising obtaining a fundus image of the patient's eye, processing the fundus image using the function obtained from the first method, and, based on the processing of the fundus image, providing an indication of the likelihood that the patient's eye has the vision-degenerative disease.

### CROSS-REFERENCE TO RELATED APPLICATIONS

This application claims the benefit of, and hereby incorporates by reference the contents of, U.S. Provisional Application No. 62/383,333, filed Sep. 2, 2016 and entitled “SCREENING METHOD FOR AUTOMATED DETECTION OF VISION-DEGENERATIVE DISEASES FROM COLOR FUNDUS IMAGES.”

### BACKGROUND

It is estimated that over 250 million people in developing regions around the world suffer from permanent vision impairment, with 80% of disease cases either preventable or curable if detected early. Thus, preventable eye diseases such as diabetic retinopathy, macular degeneration, and glaucoma continue to proliferate, causing permanent vision loss over time as they are left undiagnosed.

Current diagnostic methods are time-consuming and expensive, requiring trained clinicians to manually examine and evaluate digital color photographs of the retina, thereby leaving many patients undiagnosed and susceptible to vision loss over time. Therefore, there is an ongoing need for solutions enabling the early detection of retinal diseases to provide patients with timely access to life-altering diagnostics without dependence on medical specialists in clinical settings.

### BRIEF DESCRIPTION OF THE DRAWINGS

Objects, features, and advantages of the disclosure will be readily apparent from the following description of certain embodiments taken in conjunction with the accompanying drawings in which:

FIG 1 is a flowchart illustrating a process to generate the function F(x) by processing a dataset of fundus images in accordance with some embodiments.

FIG. 2 is a flowchart illustrating the method 100 of FIG. 1 in accordance with some embodiments.

FIG. 3 illustrates an exemplary preprocessed and pre-filtered fundus image from a dataset of fundus images after the performance of optional blocks 110 and 120 of FIG. 2.

FIG. 4 illustrates the layers of a deep learning network in accordance with some embodiments.

FIG. 5 is a flow chart that illustrates the use of the function F(x) to determine whether a patient has a vision-degenerative disease in accordance with some embodiments.

FIG. 6 illustrates a smartphone with an exemplary hardware attachment to enable the smartphone to acquire a fundus image of a patient's eye in accordance with some embodiments.

FIG. 7 illustrates a plot of extracted high-level weights from the top layer of an exemplary network.

FIG 8A illustrates an exemplary heatmap correlating to the fundus image shown in FIG. 8B, effectively highlighting large pathologies in the image.

FIG. 8B is the exemplary fundus image corresponding to the heatmap shown in FIG 8A.

FIG. 9 illustrates one example of a computer system that may be used to implement the method in accordance with some embodiments.

### DETAILED DESCRIPTION

Disclosed herein are methods and apparatuses that circumvent the need for a clinician in diagnosing vision-degenerative diseases. The methods use an automated classifier to distinguish healthy and pathological fundus (retinal) images. The disclosed embodiments provide an all-purpose solution for vision-degenerative disease detection, and the excellent results attained indicate the high efficacy of the disclosed methods in providing efficient, low-cost eye diagnostics without dependence on clinicians.

Automated solutions to current diagnostic shortfalls have been investigated in the past, but they suffer from major drawbacks that hinder transferability to clinical settings. For example, most automated algorithms derive predictive potential from small datasets of around 300 fundus images taken in isolated, singular clinical environments. These algorithms are unable to generalize to real-world applications where image acquisition will encounter artifacts, brightness variations, and other perturbations.

Based upon the need for a highly discriminative algorithm for distinguishing healthy fundus images from pathological ones, the disclosed method uses state-of-the-art deep learning algorithms. Deep learning algorithms are known to work well in computer vision applications, especially when training on large, varied datasets. By applying deep learning to a large-scale fundus image dataset representing a heterogeneous cohort of patients, the disclosed methods are capable of learning discriminative features.

In some embodiments, a method is capable of detecting symptomatic pathologies in the retina from a fundus scan. The method may be implemented on any type of device with some sort of computational power, such as a laptop or smartphone. The method utilizes state-of-the-art deep learning methods for large-scale automated feature learning to represent each input image. These features are normalized and compressed using computational techniques, for example, kernel principal component analysis (PCA), and they are fed into multiple second-level gradient-boosting decision trees to generate a final diagnosis. In some embodiments, the method reaches 95% sensitivity and 98% specificity with an area under the receiver operating characteristic AUROC of 0.97, thus demonstrating high clinical applicability for automated early detection of vision-degenerative diseases

The disclosed methods and apparatuses have a number of advantages. First, they place diagnostics in the hands of the people, eliminating dependence on clinicians for diagnostics. Individuals or technicians may use the methods disclosed herein, and devices on which those methods run, to achieve objective, independent diagnoses. Second, they reduce unnecessary workload on clinicians in medical settings; rather than spending time trying to diagnose potentially diseased patients out of a demographic of millions, clinicians can attend to patients already determined to be at high-risk for a vision loss disease, thereby focusing on providing actual treatment in a time-efficient manner.

In some embodiments, a large set of fundus images representing a variety of eye conditions (e.g., healthy, diseased) is processed using deep learning techniques to determine a function, F(x). The function F(x) may then be provided to an application on a computational device (e.g., a computer (laptop, desktop, etc.) or a mobile device (smartphone, tablet, etc.)), which may be used in the field to diagnose patients' eye diseases. In some embodiments, the computational device is a portable device that is fitted with hardware to enable the portable device to take a fundus image of the eye of a patient who is being tested for eye diseases, and then the application on the portable device processes this fundus image using the function F(x) to determine a diagnosis. In other embodiments, a previously-taken fundus image of the patient's eye is provided to a computational device (e.g., any computational device, whether portable or not), and the application processes the fundus image using the function F(x) to determine a diagnosis.

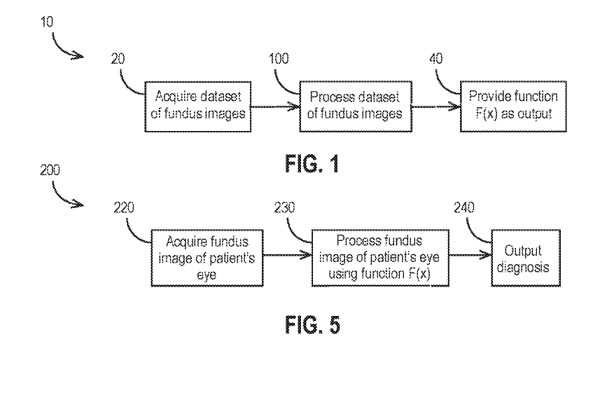

FIG. 1 is a flowchart 10 illustrating a process to generate the function F(x) by processing a dataset of fundus images in accordance with sonic embodiments. At block 20, a dataset of RGB (red, green, blue) fundus images is acquired. Preferably, the dataset includes many images (e.g., 102, 514 color fundus images), containing a wide variety of image cases (e.g., taken under a variety of lighting conditions, taken using a variety of camera models, representing a variety of eye diseases, representing a variety of ethnicities, representing various parts of the retina as well (not simply the fundus or not the fundus specifically), etc.). For example the dataset may contain a comprehensive set of fundus images from patients of different ethnicities taken with varying camera models. The dataset may be obtained from, for example, public datasets and/or eye clinics. To preserve patient confidentiality, the images may be received in a de-identified format without any patient identification.

Preferably, the images in the dataset represent a heterogeneous cohort of patients with a multitude of retinal afflictions indicative of various ophthalmic diseases, such as, for example, diabetic retinopathy, macular edema, glaucoma, and age-related macular degeneration. Each of the input images in the dataset has been pre-associated with a diagnostic label of “healthy” or “diseased.” For example, the diagnostic labels may have been determined by a panel of medical specialists. These diagnostic labels may be any convenient labels, including alphanumeric characters. For example, the labels may be numerical, such a value of 0 or 1, where 0 is healthy and 1 is diseased, or a value, possibly non-integer, possibly in a range between a minimum value and a maximum value (e.g., in range of [0-5], which is simply one example) to represent a continuous risk descriptor. Alternatively or in addition, the labels may include letters or other indicators.

At block 100, representing a method 100, the dataset of fundus images is processed in accordance with the novel methods disclosed herein, discussed in more detail below. At block 40, the function F(x), which may be used thereafter to diagnose vision-degenerative diseases as described in more detail below, is provided as an output.

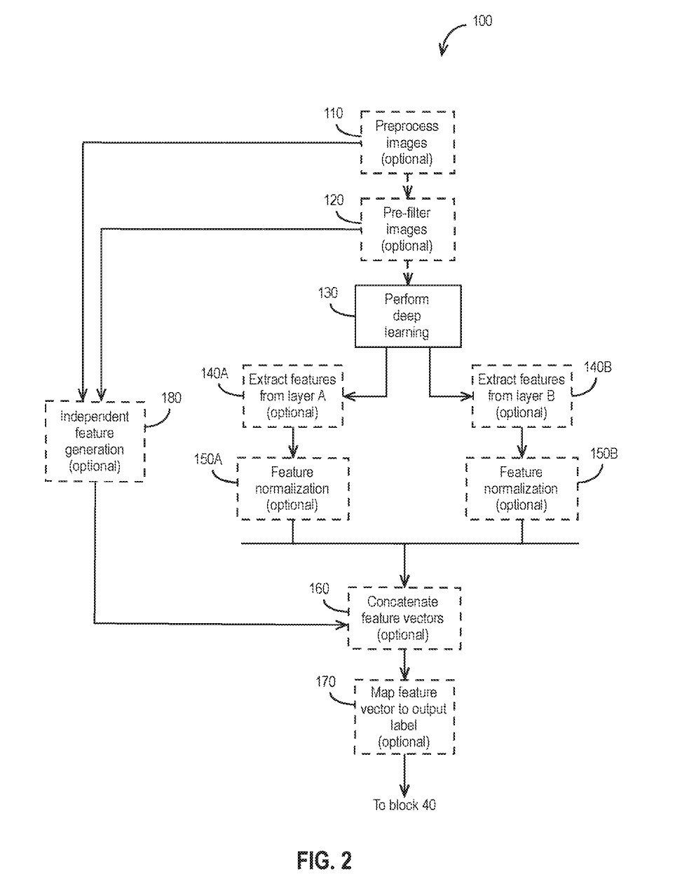

FIG. 2 is a flowchart illustrating the method 100 of FIG. 1 in accordance with some embodiments. At block 110, the images in the dataset of fundus images are optionally preprocessed. If performed, the preprocessing of block 110 may improve the resulting processing performed in the remaining blocks of FIG. 2. In some embodiments that include block 110, each input fundus image is preprocessed by normalizing pixel values using a conventional algorithm, such as, for example, L2 normalization. In some embodiments that include block 110 the pixel RGB channel distributions are normalized by subtracting the mean and standard deviation images to generate a single preprocessed image from the original unprocessed image. This step may aid in end accuracy of the model. Other potential preprocessing optionally performed at block 110 may include applying contrast enhancement for enhanced image sharpness, and/or resizing each image to a selected size (e.g., 512×512 pixels) to accelerate processing. In some embodiments that include block 110, contrast enhancement is achieved using contrast-limited adaptive histogram equalization (CLAHE). In some embodiments that include block 110, the image is resized using conventional bilinear interpolation. There are many ways by which the optional preprocessing of block 110 may be accomplished, and the examples provided herein are not intended to be limiting. As stated above, the preprocessing of block 110 is optional but may improve accuracy.

At block 120, the images in the dataset are optionally pre-filtered. If used, pre-filtering may result in the benefit of encoding robust invariances into the method, or it may enhance the final accuracy of the model. In some embodiments using pre-filtering, each image is rotated by some random number of degrees (or radians) using any computer randomizing technique (e.g., by using a pseudo-random number generator to choose the number of degrees/radians by which each image is rotated). In some embodiments that include block 120, each image is randomly flipped horizontally (e.g., by randomly selecting a value of 0 or 1, where 0 (or 1) means to flip the image horizontally and 1 (or 0) means not to flip the image horizontally). In some embodiments that include block 120, each image is randomly flipped vertically (e.g., by randomly selecting a value of 0 or 1, where 0 (or 1) means to flip the image vertically and 1 (or 0) means not to flip the image vertically). In some embodiments that include block 120, each image is skewed using conventional image processing techniques in order to account for real-world artifacts and brightness fluctuations that may arise during image acquisition with a smartphone camera. The examples provided herein are some of the many transformations that may be used to pre-filer each image, artificially augmenting the original dataset with variations of perturbed images. Other pre-filtering techniques may also or alternatively be used in the optional block 1202, and the examples given herein are not intended to be limiting.

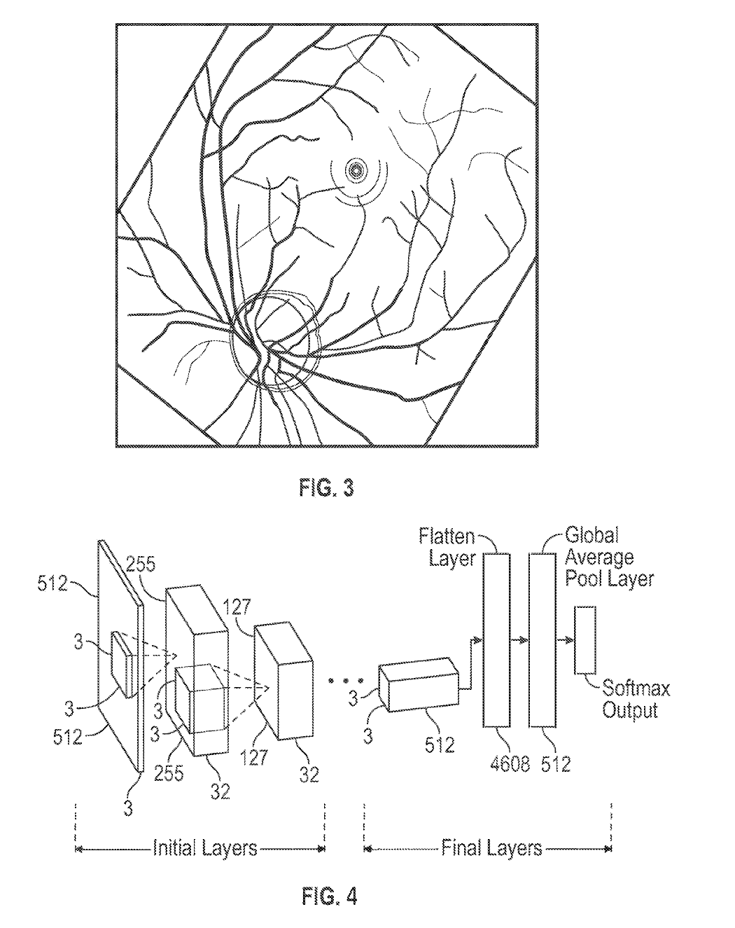

FIG. 3 shows an exemplary preprocessed and pre-filtered fundus image from a dataset of fundus images after the performance of optional blocks 110 and 120. In the example of FIG. 3, the image was preprocessed by subtracting the mean and standard deviation images from the original unprocessed image, applying contrast enhancement using CLAHE, and resizing the image. The image was pre-filtered by randomly rotating, horizontally flipping, and skewing the image.

Referring again to FIG. 2, at block 130, the images, possibly preprocessed and/or pre-filtered at blocks 110 and/or 120, are fed into a custom deep learning neural network that performs deep learning. In some embodiments, the custom deep learning network is a residual convolutional neural network, and the method performs deep learning using the residual convolutional neural network to learn thorough features for discriminative separation of healthy and pathological images. Convolutional neural networks are state-of-the-art image-recognition techniques that have wide applicability in image recognition tasks. These networks may be represented by composing together many different functions. As used by at least some of the embodiments disclosed herein, they use convolutional parameter layers to iteratively learn filters that transform input images into hierarchical feature maps, learning discriminative features at varying spatial levels.

For example, there may be N functions, denoted as g1, g2, g3, . . . , gN connected in a chain, to form g(x)=gN( . . . (g3(g2(g1(x))), where g1 is called the first layer of the network, g2 is called the second layer, and so on. The depth of the model is the number of functions in the chain. Thus, for the example given above, the depth of the model is N. The final layer of the network is called the output layer, and the other layers are called hidden layers. The learning algorithm decides how to use the hidden layers to produce the desired output. Each hidden layer of the network is typically vector-valued. The width of the model is determined by the dimensionality of the hidden layers.

In some embodiments, the input is presented at the layer known as the “visible layer.” A series of hidden layers then extracts features from an input image. These layers are “hidden” because the model determines which concepts are useful for explaining relationships in the observed data.

In some embodiments, a custom deep convolutional network uses the principle of “residual-learning,” which introduces identity-connections between convolutional layers to enable incremental learning of an underlying polynomial function. This may aid in final accuracy, but residual learning is optional—any variation of a neural network (preferably convolutional neural networks in some form with sufficient depth for enhanced learning power) may be used in the embodiments disclosed herein.

In some embodiments, the custom deep convolutional network contains many hidden layers and millions of parameters. For example, in one embodiment, the network has 26 hidden layers with a total of 6 million parameters. The deep learning textbook entitled “Deep Learning,” by Ian Goodfellow, Yoshua Bengio, and Aaron Courville (MIT Press), available Online at http://www.deeplearningbook.org, provides information about how neural networks and convolutional variants work and is hereby incorporated by reference. FIG. 4 illustrates the layers of the network in accordance with some embodiments.

Referring again to FIG. 2, at optional blocks 140A and 140B, intermediate features from the convolutional neural network are extracted from selected layers of the network (referred to as “layer A” and “layer B”) as a feature vector. Each feature vector is a vector of numbers corresponding to the output of a selected layer. The term “features” refers to the output of a neural network layer (refer to http://www.deeplearningbook.org/). In some embodiments, intermediate features from a single layer (e.g., from layer A or layer B ) are extracted from the neural network. In some embodiments, intermediate features from multiple layers (e.g., from layer A and layer B) are extracted from the neural network. In some embodiments, including as illustrated in FIG. 2, intermediate features from two selected layers are extracted from the neural network. In some embodiments in which intermediate features from two selected layers are extracted, the two layers are the penultimate layer and final convolutional layer. However, any layer (for embodiments using a single layer) or layers (for embodiments using multiple layers) may suffice to extract features from the model to best describe each input image. In some embodiments, the layers are the global average pooling layer (layer B) and the final convolutional layer (layer A), yielding two bags of 512 features and 4608 features, respectively. In some embodiments, extracting features from the last layer of the network corresponds to a diagnosis itself, mapping to the original label that was defined per image of the dataset used to train the network. This approach can provide an output label sufficient as a diagnosis. In other embodiments, however, for increased diagnosis accuracy, a second-level classifier is included that uses information from the network and from outside computations, as described below. Use of the second-level classifier can help in accuracy in some variants, as it includes more information when creating a diagnosis (for example, statistical image features, handcrafted features capturing describing variant biological phenomena in the fundus etc.).

At blocks 150A and 150B, the extracted features are optionally normalized and/or compressed. In some embodiments including one or both of blocks 150A and 150B, the features from both layer A and layer B are normalized. For example, in embodiments using the last convolutional layer and the global average pool layer, the features may be normalized using L2 normalization to restrict the values to between [0,1]. If used, the normalization may be, achieved by any normalization technique, L2 normalization being just one non-limiting example. As indicated in FIG. 2, feature normalization is optional, but it may aid in final model accuracy.

In addition or alternatively, at blocks 150A and 150B, the features may be compressed. For example, a kernel PCA function may optionally be used (e.g., on the features from the last convolutional layer) to map the feature vector to a smaller number of features (e.g., 1034 features) in order to enhance feature correlation before decision tree classification. The use of a PCA function may improve accuracy. For example, a kernel PCA may be used to map the feature vector of the last convolutional layer to a smaller number of features. Kernel PCA is just one option out of many compression algorithms that may be used to map a large number of features to a smaller number of features. Any compression algorithm may be alternatively be used (e.g., independent component analysis (ICA), non-kernel PCA, etc.). As indicated in FIG. 2, both normalization and compression are optional but may be helpful in some algorithm variants for accuracy.

As illustrated in FIG. 2, independent feature generation (e.g., statistical feature extraction, handcrafted feature extraction, or any other type of feature generation) may optionally be used at block 180 to improve accuracy. As illustrated in FIG. 2, if included, independent feature generation at block 180 may be performed on preprocessed images emerging from image preprocessing at block 110 (if included) or on pre-filtered images emerging from image pre-filtering at block 120 (if included). Alternatively, independent feature generation may optionally be performed on images from the original image dataset.

One type of independent feature generation is statistical feature extraction. Thus, in addition to automated feature extraction with deep learning, thorough statistical feature extraction using conventional filters in the computer vision field may be used at optional block 180. In contrast to the fine-tuned filters learned through the deep learning training process, conventional filters may optionally be used for supplemental feature extraction to enhance accuracy. For example, statistical feature extraction may be performed using any of Riesz features, co-occurrence matrix features, skewness, kurtosis, and/or entropy statistics. In experiments performed by the inventor using Riesz features, co-occurrence matrix features, skewness, kurtosis, and entropy statistics, these features formed a final feature vector of 56 features. It is to be understood that there are many ways to perform statistical feature extraction, and the examples given herein are provided to illustrate but not limit the scope of the claims.

Another type of independent feature generation is handcrafted feature extraction. Thus, in addition to automated feature extraction with deep learning, handcrafted feature extraction may be utilized to describe an image. One may handcraft filters (as opposed to those automatically generated within the layers of deep learning) to specifically generate feature vectors that represent targeted phenomena in the image, (e.g., a micro-aneurysm (a blood leakage from a vessel), an exudate (a plaque leakage from a vessel), a hemorrhage (large blood pooling out of a vessel), the blood vessel itself, etc.). It is to be understood that there are many ways to perform handcrafted feature extraction (for example, constructing Gabor filter banks), and the examples given herein are provided to illustrate but not limit the scope of the claims. A person having ordinary skill in the art would understand the use of conventional methods of image discrimination and representation that would be useful in the scope of the disclosures herein.

The features extracted from the neural network (e.g., directly from blocks 140A and 140B, or from optional blocks 150A and 150B, if present) and, if present, independently generated features from optional block 180 are combined and fed into optional block 160, which concatenates feature vectors. In some embodiments, the feature vector concatenation is accomplished using a gradient boosting classifier, with the input being a long numerical vector (or multiple long numerical vectors) with the training label being the original diagnostic label.

At block 170, feature vectors are mapped to output labels. In some embodiments, the output labels are numerical in the form of the defined diagnostic label (e.g., 0 or 1, continuous variable between a minimum value and a maximum value (e.g., 0 to 5), etc.). This may be interpreted in many ways, such as by thresholding at various levels to optimize metrics such as sensitivity, specificity, etc. In some embodiments, thresholding at 0.5 with a single numerical output may provide adequate accuracy.

In some embodiments, the feature vectors are mapped to output labels by performing gradient-boosting decision-tree classification. In some such embodiments, separate gradient-boosting classifiers are optionally used separately on each bag of features. Gradient-boosting classifiers are tree-based classifiers known for capturing fine-grained correlations in input features based on intrinsic tree-ensembles and bagging. In some embodiments, the prediction from each classifier is weighted using standard grid-search to generate a final diagnosis Score. Grid search is a way for computers to determine optimal parameters. Grid search is optional but may improve accuracy. The use of gradient-boosting classifiers is also optional; any supervised learning algorithm that can map feature vectors to an output label may work, such as the Support Vector Machine classification or Random Forest classification, Gradient-boosting classifiers may have better accuracy than other candidate approaches, however. A person having ordinary skill in the art would understand the use of conventional methods to map feature vectors to corresponding labels that would be useful in the scope of the disclosures herein.

The output labels from blocks 160A and 160B are then provided to block 40 of FIG. 1. Referring again to FIG. 1, the function F(x) is provided as an output. The function F(x) may be stored on a computer, such as a desktop or laptop computer, or it may be stored on a mobile device, such as a smartphone.

It is to be understood that FIG. 2 illustrates one specific embodiment of the method performed in block 100 of FIG. 1. Variations are possible and are within the scope of this disclosure. For example, although it may be advantageous to perform at least some of the optional blocks 110, 120, 140A, 140B, 150A, 150B, 160, 170, 180, it is within the scope of the disclosure to perform none of the optional blocks shown in FIG. 2. In some such embodiments, only the block 130 (perform deep learning) is performed, and the last layer of the neural network, which describes the fully deep-learning-mapped vector, is used as the final output.

As another example, instead of processing both the illustrated left-hand branch (blocks 140A and 150A) and the right-hand branch (blocks 140B and 150B), one of the branches may be eliminated altogether. Furthermore, although FIG. 2 illustrates an embodiment in which the last convolutional layer and the global average pool layer are used, other layers from the deep learning network may be used instead. Thus, the scope of this disclosure includes embodiments in which a single selected layer of the deep learning network is used, where the selected layer may be any suitable layer, such as the last convolutional layer, the global average pool layer, or another selected layer. All such embodiments are within the scope of the disclosures herein.

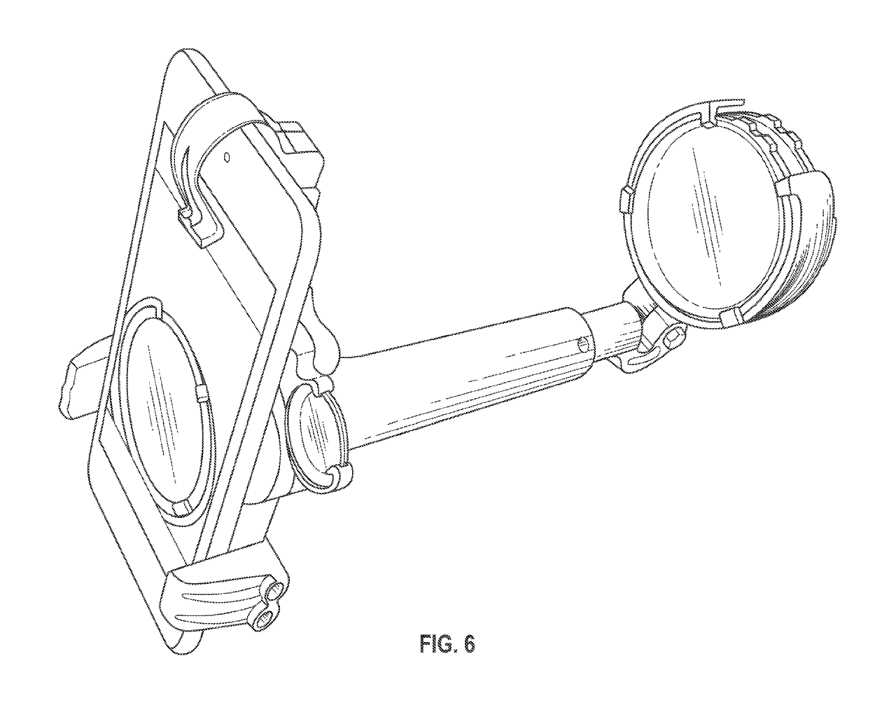

FIG. 5 is a flowchart 200 that illustrates the use of the function F(x) to determine whether a patient has a vision-degenerative disease in accordance with some embodiments. At block 220, a fundus image of a patient's eye is acquired. The fundus image may be acquired, for example, by attaching imaging hardware to a mobile device. FIG. 6 illustrates a smartphone with an exemplary hardware attachment to enable the smartphone to acquire a fundus image of a patient's eye. As another example, the fundus image of the patient's eye may be acquired in some other way, such as from a database or another piece of imaging equipment, and provided to the device (e.g., computer or mobile device) performing the diagnosis.

At block 230, the fundus image of the patient's eye is processed using the function F(x). For example, an app on a smartphone may process the fundus image of the patient's eye. At block 240, a diagnosis is provided as output. For example, the app on the smartphone may provide a diagnosis that indicates whether the analysis of the fundus image of the patient's eye suggests that the patient is suffering from a vision-degenerative disease.

An embodiment of the disclosed method has been tested using five-fold stratified cross-validation, preserving the percentage of samples of each class per fold. This testing procedure split the training data into five buckets of around 20,500 images. The method trained on four folds and predicted the labels of the remaining one, repeating this process once per fold. This process ensured model validity independent of the specific partition of training data used.

The implemented embodiment derived average metrics from five test runs by comparing the embodiment's predictions to the gold standard determined by a panel of specialists. Two metrics were chosen to validate the embodiment:

Area Under the Receiver Operating Characteristic (AUROC) curve: The receiver operating characteristic (ROC) curve is a graphical plot that illustrates the performance of a binary classifier by measuring the tradeoff between its true positive and false positive rates. The closer the area under this curve is to 1, the smaller the tradeoff, indicating greater predictive potential. The implemented embodiment scored an average AUROC of 0.97 during 5-fold cross-validation. This metric is a near perfect result, indicating excellent performance on a large-scale dataset.

Sensitivity and Specificity: Sensitivity and specificity indicate the rate of true positive cases among all classifications, whereas specificity measures the rate of true negatives. As indicated by Table 1 below, the implemented embodiment achieved an average 95% sensitivity and a 98% specificity during 5-fold cross validation. This statistic represents the highest point on the ROC curve with minimal tradeoff between precision and recall.

TABLE 1
Sensitivity, specificity, and AUROC values per fold and on average

| Metric | Sensitivity | Specificity | AUROC |
|---|---|---|---|
| Fold 1 | 0.94 | 0.97 | 0.96 |
| Fold 2 | 0.96 | 0.98 | 0.98 |
| Fold 3 | 0.97 | 0.97 | 0.96 |
| Fold 4 | 0.93 | 0.99 | 0.95 |
| Fold 5 | 0.95 | 0.97 | 0.97 |
| Average value | 0.95 | 0.98 | 0.97 |

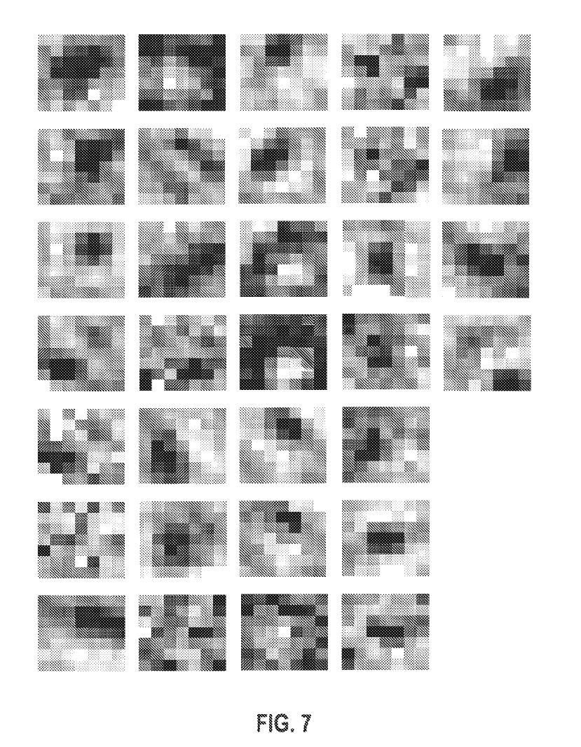

To validate the efficiency of the residual network in learning highly discriminative filters for optimal feature map generation, FIG. 7 shows a plot of the extracted high-level weights from the top layer of the network. FIG. 7 contrast-normalizes each filter for better visualization. Note the fine-grained details encoded in each filter based on the iterative training cycle of the neural network. These filters look highly specific in contrast to more general computer vision filters, such as Gabor filters.

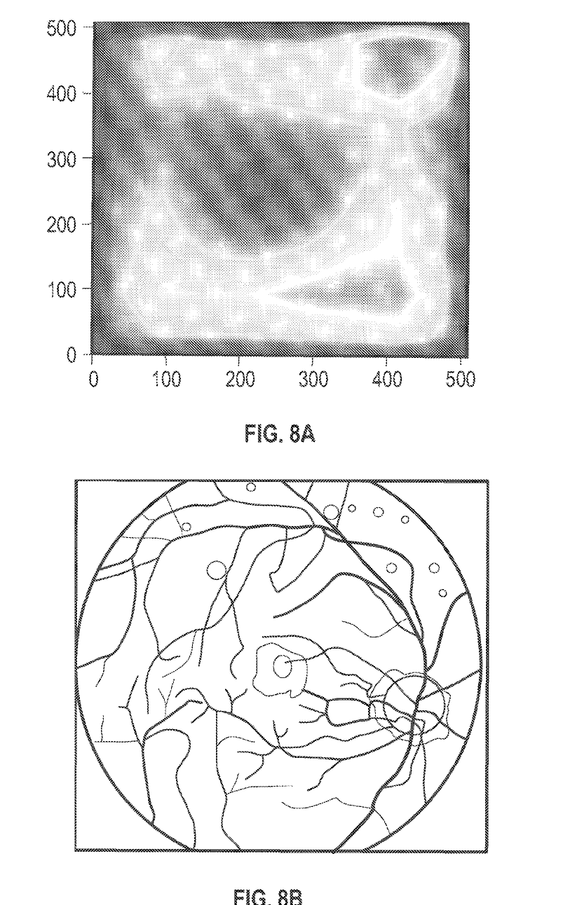

To validate the prognostic performance of the implemented embodiment, an occlusion heatmap was generated on sample pathological fundus images. This heatmap was generated by occluding parts Of an input image iteratively, and highlighting regions of the image that greatly impact the diagnostic output in red while highlighting irrelevant regions in blue. FIG. 8A shows a version of a sample heatmap correlating to the fundus image shown in FIG. 8B, effectively highlighting large pathologies in the image. This may also be provided as an output to the user, highlighting pathologies in the image for further diagnosis and analysis.

As explained previously, the disclosed methods may be implemented in a portable apparatus. For example, an apparatus for vision-degenerative disease detection comprises an external lens attached to a smartphone that implements the disclosed method. The smartphone may include an application that implements the disclosed method. The apparatus provides rapid, portable screening for vision-degenerative diseases, greatly expanding access to eye diagnostics in rural regions that would otherwise lack basic eye care. Individuals are no longer required to seek out expensive medical attention each time they wish for a retinal evaluation, and can instead simply use the disclosed apparatus for efficient evaluation.

The efficacy of the disclosed method was tested in an iOS smartphone application built using Swift and used in conjunction with a lens attached to the smartphone. This implementation of one embodiment was efficient in diagnosing input retinal scans, taking on average 10 seconds to generate a diagnosis. The application produced a diagnosis in approximately 8 seconds in real-time and was tested on an iPhone 5.

For proper clinical application, further testing and optimization of the sensitivity metric may be necessary in order to ensure minimum false negative rates. In order to further increase the sensitivity metric, it may be important to control specific variances in the dataset, such as ethnicity or age, to optimize our algorithm for certain demographics during deployment.

The disclosed method may be implemented on a computer programmed to execute a set of machine-executable instructions. In some embodiments, the machine-executable instructions are generated from computer code written in the Python programming language, although any suitable computer programming language may be used instead.

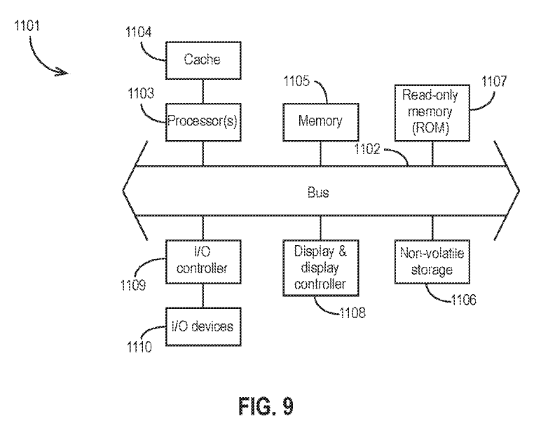

FIG. 9 shows one example of a computer system that may be used to implement the method 100. Note that although FIG. 9 illustrates various components of a computer system, it is not intended to represent any particular architecture or manner of interconnecting the components, as such details are not germane to the present disclosure. It should be noted that the architecture of FIG. 9 is provided for purposes of illustration only and that a computer system or other digital processing system used to implement the embodiments disclosed herein is not limited to this specific architecture. It will also be appreciated that network computers and other data processing systems that have fewer components or perhaps more components may also be used with the embodiments disclosed herein. The computer system of FIG. 9 may, for example, be a server or a desktop computer running any suitable operating system (e.g., Microsoft Windows, Mac OS, Linux, Unix, etc.). Alternatively, the computer system of FIG. 9 may be a mobile or stationary computational device, such as, for example, a smartphone, a tablet, a laptop, or a desktop computer or Server.

As shown in FIG. 9, the computer system 1101, which is a form of a data processing system, includes a bus 1102 that is coupled to a microprocessor 1103 and a ROM 1107 and volatile RAM 1105 and a non-volatile memory 1106. The microprocessor 1103, which may be a microprocessor from Intel or Motorola, Inc. or IBM, is coupled to cache memory 1104. The bus 1102 interconnects these various components together and may also interconnect the components 1103, 1107, 1105, and 1106 to a display controller and display device 1108 and to peripheral devices such as input/output (I/O) devices, which may be mice, keyboards, modems, network interfaces, printers, scanners, displays (e.g., cathode ray tube (CRT) or liquid crystal display (LCD)), video cameras, and other devices that are well known in the art. Typically, the input/output devices 1110 are coupled to the system through input/output controllers 1109.

Output devices may include, for example, a visual output device, an audio output device, and/or tactile output device (e.g., vibrations, etc.). Input devices may include, for example, an alphanumeric input device, such as a keyboard including alphanumeric and other keys, for enabling a user to communicate information and command selections to the microprocessor 1103. Input devices may include, for example, a cursor control device, such as a mouse, a trackball, stylus, cursor direction keys, or touch screen, for communicating direction information and command selections to the microprocessor 1103, and for controlling movement on the display & display controller 1108.

The I/O devices 1110 may also include a network device for accessing other nodes of a distributed system via the communication network 116. The network device may include any of a number of commercially available networking peripheral devices such as those used for coupling to an Ethernet, token ring, Internet, or wide area network, personal area network, wireless network, or other method of accessing other devices. The network device may further be a null-modem connection, or any other mechanism that provides connectivity to the outside world.

The volatile RAM 1105 may implemented as dynamic RAM (DRAM), which requires power continually in order to refresh or maintain the data in the memory. The non-volatile memory 1106 may be a magnetic hard drive or a magnetic optical drive or an optical drive or a DVD RAM or other type of memory system that maintains data even after power is removed from the system. Typically, the nonvolatile memory will also be a random access memory, although this is not required. Although FIG. 9 shows that the non-volatile memory is a local device coupled directly to the rest of the components in the data processing system, it will be appreciated that the system may utilize a non-volatile memory that is remote from the system, such as a network storage device that is coupled to the data processing system through a network interface such as a modem or Ethernet interface. The bus 1102 may include one or more buses connected to each other through various bridges, controllers and/or adapters as is well known in the art. The I/O controller 1109 may include a USB (Universal Serial Bus) adapter for controlling USB peripherals, and/or an IEEE 1394 bus adapter for controlling IEEE-1394 peripherals.

It will be apparent from this description that aspects of the method 100 may be embodied, at least in part, in software. That is, the techniques may be carried out in a computer system or other data processing system in response to its processor, such as a microprocessor, executing sequences of instructions contained in a memory, such as ROM 1107, volatile RAM 1105, non-volatile memory 1106, cache 1104 or a remote storage device. In various embodiments, hard-wired circuitry may be used in combination with software instructions to implement the method 100. Thus, the techniques are not limited to any specific combination of hardware circuitry and software, nor to any particular source for the instructions executed by the data processing system. In addition, various functions and operations may be performed by or caused by software code, and therefore the functions and operations result from execution of the code by a processor, such as the microprocessor 1103.

A non-transitory machine-readable medium can be used to store software and data (e.g., machine-executable instructions) that, when executed by a data processing system (e.g., at least one processor), causes the system to perform various methods disclosed herein. This executable software and data may be stored in various places including for example ROM 1107, volatile RAM 1105, non-volatile memory 1106 and/or cache 1104. Portions of this software and/or data may be stored in any one of these storage devices.

Thus, a machine-readable medium includes any mechanism that provides (i.e., stores and/or transmits) information in a form accessible by a machine (e.g., a computer, network device, mobile device, personal digital assistant, manufacturing tool, any device with a set of one or more processors, etc.). For example, a machine-readable medium includes recordable/non-recordable media (e.g., read only memory (ROM), random access memory (RAM), magnetic disk storage media, optical storage media, flash memory devices, etc.).

Note that any of all of the components of this system illustrated in FIG. 9 and associated hardware may be used in various embodiments. It will be appreciated by those of ordinary skill in the art that the particular machine that embodies the method 100 may be configured in various ways according to the particular implementation. The control logic or software implementing the disclosed embodiments can be stored in main memory, a mass storage device, or other storage medium locally or remotely accessible to processor 1103 (e.g., memory 125 illustrated in FIG. 2).

In the foregoing description and in the accompanying drawings, specific terminology has been set forth to provide a thorough understanding of the disclosed embodiments. In some instances, the terminology or drawings may imply specific details that are not required to practice the disclosed embodiments.

To avoid obscuring the present disclosure unnecessarily, well-known components (e.g., memory) are shown in block diagram form and/or are not discussed in detail or, in some cases, at all.

Unless otherwise specifically defined herein, all terms are to be given their broadest possible interpretation, including meanings implied from the specification and drawings and meanings understood by those skilled in the art and/or as defined in dictionaries, treatises, etc. As set forth explicitly herein, some terms may not comport with their ordinary or customary meanings.

As used in the specification and the appended claims, the singular forms “a,” “an” and “the” do not exclude plural referents unless otherwise specified. The word “or” is to be interpreted as inclusive unless otherwise specified. Thus, the phrase “A or B” is to be interpreted as meaning all of the following: “both A and B,” “A but not B,” and “B but not A.” Any use of “and/or” herein does not mean that the word “or” alone connotes exclusivity.

As used herein, phrases of the form “at least one of A, B, and C,” “at least one of A, B, or C,” “one or more of A, B, or C,” and “one or more of A, B and C” are interchangeable, and each encompasses all of the following meanings: “A only,” “B only,” “C only,” “A and B but not C,” “A and C but not B,” “B and C but not A,” and “all of A, B, and C.”

To the extent that the terms “include(s),” “having,” “has,” “with,” and variants thereof are used in the detailed description or the claims, such terms are intended to be inclusive in a mariner similar to the term “comprising,” i.e., meaning “including but not limited to.” The terms “exemplary” and “embodiment” are used to express examples, not preferences or requirements.

Although specific embodiments have been disclosed, it will be evident that various modifications and changes may be made thereto without departing from the broader spirit and scope of the disclosure. For example, features or aspects of any of the embodiments may be applied, at least where practicable, in combination with any other of the embodiments or in place of counterpart features or aspects thereof. Accordingly, the specification and drawings are to be regarded in an illustrative rather than a restrictive sense.

1. A method of determining a function for use in diagnosing a vision-degenerative disease, the method comprising:
obtaining a dataset of fundus images;
using a custom deep learning network to process the dataset of fundus images; and
providing the function as an output.

2. The method of claim 1, wherein the dataset of fundus images comprises at least 500 images.

3. The method of claim 1, wherein each of the fundus images in the dataset is associated with a diagnostic label.

4. The method of claim 3, wherein the diagnostic label is a value.

5. The method of claim 4, wherein the value is a first value or a second value, the first value indicating a healthy eye and the second value indicating a diseased eye.

6. The method of claim 4, wherein the value is selected from a continuum of values between a first value and a second value.

7. The method of claim 6, wherein the value indicates a probability or risk of disease.

8. The method of claim 4, wherein the value is an integer.

9. The method of claim 4, wherein the value is a real number.

10. The method of claim 4, wherein the value is alphanumeric.

11. The method of claim 1, wherein the custom deep learning network is a residual convolutional neural network.

12. The method of claim 11, wherein using the custom deep learning network to process the dataset of fundus images comprises performing deep residual learning using the residual convolutional neural network to learn features enabling discrimination of healthy and pathological images from the dataset of fundus images.

13. The method of claim 11, wherein using the custom deep learning network to process the dataset of fundus images comprises performing deep residual learning using the network to directly map an input image from the dataset of fundus images to a predetermined label enabling discrimination of healthy and pathological images from the dataset of fundus images.

14. The method of claim 11, further comprising:
mapping an image from the dataset of fundus images to a predetermined label enabling discrimination of healthy and pathological images from the dataset of fundus images.

15. The method of claim 1, wherein the custom deep learning network comprises at least 20 layers and at least 1 million parameters.

16. The method of claim 1, wherein using the custom deep learning network to process the dataset of fundus images comprises extracting intermediate features from a penultimate layer and a final layer.

17. The method of claim 1, wherein using the custom deep learning network to process the dataset of fundus images comprises extracting intermediate features from a first selected layer and a second selected layer.

18. The method of claim 1, further comprising:
extracting at least one feature.

19. The method of claim 18, further comprising:
normalizing the at least one feature.

20. The method of claim 19, wherein normalizing the at least one feature comprises performing L2 normalization.

21. The method of claim 18, further comprising:
compressing the at least one feature.

22. The method of claim 21, wherein the at least one feature comprises a feature vector of a selected layer, and wherein compressing the at least one feature comprises:
using a kernel PCA to map the feature vector to a smaller number of features.

23. The method of claim 21, wherein the at least one feature comprises a plurality of features, and wherein compressing the at least one feature comprises:
mapping the plurality of features to a smaller number of features.

24. The method of claim 21, wherein compressing the at least one feature comprises performing independent component analysis (ICA) or non-kernel PCA.

25. The method of claim 18, further comprising:
extracting at least one statistical feature.

26. The method of claim 25, wherein extracting at least one statistical feature comprises using a Riesz feature, a co-occurrence matrix feature, skewness, kurtosis, or an entropy statistic.

27. The method of claim 18, further comprising:
generating at least one independent feature.

28. The method of claim 27, wherein generating the at least one independent feature comprises performing handcrafted feature extraction.

29. The method of claim 28, wherein performing handcrafted feature extraction comprises:
configuring a filler; and
generating a feature vector, wherein the feature vector represents a particular phenomenon.

30. The method of claim 29, wherein the particular phenomenon is a micro-aneurysm, hemorrhage, exudate, or blood vessel.

31. The method of claim 29, wherein the configuring a filter is achieved through a Gabor filter bank.

32. The method of claim 1, wherein using the deep learning network to process the dataset of fundus images comprises mapping a feature vector to an output label.

33. The method of claim 32, wherein mapping the feature vector to the output label comprises:
performing a classification.

34. The method of claim 33, wherein the classification is a gradient-boosting decision-tree classification.

35. The method of claim 33, wherein the classification is a Support Vector Machine classification.

36. The method of claim 33, wherein the classification is a Random Forest classification.

37. The method of claim 33, wherein performing the classification comprises performing a grid search.

38. The method of claim 1, further comprising preprocessing at least a portion of the dataset of fundus images before using the custom deep learning network to process the dataset of fundus images.

39. The method of claim 38, wherein preprocessing the at least a portion of the dataset of fundus images comprises normalizing a pixel value in the at least a portion of the dataset of fundus images.

40. The method of claim 39. wherein normalizing the pixel value comprises performing L2 normalization.

41. The method of claim 39, wherein normalizing the pixel value comprises subtracting a mean and a standard deviation image from an original fundus image in the at least a portion of the dataset of fundus images, applying contrast enhancement to a selected fundus image from the at least a portion of the dataset of fundus images, or resizing the selected fundus image from the at least a portion of the dataset of fundus images.

42. The method of claim 1, further comprising pre-filtering at least a portion of the dataset of fundus images before using the custom deep learning network to process the dataset of fundus images or extracting information using statistical or handcrafted features.

43. The method of claim 42, wherein pre-filtering the at least a portion of the dataset of fundus images comprises randomly rotating the at least a portion of the dataset of fundus images, randomly flipping the at least a portion of the dataset of fundus images, or skewing the at least a portion of the dataset of fundus images.

44. The method of claim 1, further comprising:
providing the function to a portable device.

45. The method of claim 44, wherein the portable device comprises a smartphone, a tablet, or a laptop computer.

46. The method of claim 44, wherein the portable device includes an application configured to use the function to diagnose whether a patient's eye has the vision-degenerative disease.

47. A non-transitory computer-readable storage medium storing machine-executable instructions that, when executed by one or more processors, cause the one or more processors to perform the method recited in claim 1.

48. A computing device comprising one or more processors configured to perform the method recited in claim 1.

49. A computing device comprising:
memory storing a representation of the function produced by the method of claim 1; and
one or more processors configured to use the function to assist in the diagnosis of the vision-degenerative disease.

50. A method of determining a likelihood that a patient's eye has the vision-degenerative disease, the method comprising:
obtaining a fundus image of the patient's eye;
processing the fundus image of the patient's eye using the function obtained from the method of claim 1; and
based on the processing of the fundus image of the patient's eye, providing an indication of the likelihood that the patient's eye has the vision-degenerative disease.

51. The method of claim 50, further comprising providing a heatmap identifying a location of a possible pathology in the patient's eye.

52. The method of claim 50, wherein processing the fundus image of the patient's eye comprises launching an application on a portable device, the portable device having a camera, and wherein obtaining the fundus image of the patient's eye comprises:
attaching a lens to the portable device; and
capturing the fundus image using the lens and the camera.

53. The method of claim 52, wherein the portable device comprises a smartphone, a tablet, or a laptop computer.

54. The method of claim 50, wherein processing the fundus image of the patient's eye comprises launching an application on a portable device, and wherein providing the indication of the likelihood that the patient's eye has the vision-degenerative disease comprises the application providing the indication.

55. A non-transitory computer-readable storage medium storing machine-executable instructions that, when executed by one or more processors, cause the one or more processors to perform the method recited in claim 50.

56. A computing device comprising one or more processors configured to perform the method recited in claim 50.
